(19) United States
(12) Patent Application Publication
(10) Pub. No.: US 2014/0340636 A1
(43) Pub. Date: Nov. 20, 2014

(54) METHOD AND DEVICE FOR MEASURING HAEMOGLOBIN IN THE EYE

(71) Applicant: INSTRUMENTACTION Y OFTALMOLOGIA INSOFT S.L., Sta. Cruz Tenerife (ES)

(72) Inventor: Manuel Gonzalez De La Rosa, Sta. Cruz de Tenerife (ES)

(21) Appl. No.: 14/360,548

(22) PCT Filed: Nov. 21, 2012
(86) PCT No.: PCT/ES2012/070810

§ 371 Date: May 23, 2014

(30) Foreign Application Priority Data
Nov. 23, 2011 (ES) P201131884

(51) Int. Cl. A61B 3/12; A61B 3/14; A61B 3/00; A61B 5/1455

(52) U.S. Cl. CPC A61B 3/1241; A61B 5/1455; A61B 3/14; A61B 3/0025
USPC 351/206; 600/320

(57) ABSTRACT

Based on the identification by colorimetry of a colour image of the fundus of the eye of an individual, obtained by specialised cameras (fundus camera or retinograph). The method and the device of the present invention enable establishing a direct relation between the chromatic properties of the various tissues of the optic nerve (10) and the amount of haemoglobin (Hb) in the optic nerve head (10), allowing identification and clearly distinguishing the irrigated regions of the optic nerve (10) from the non-irrigated regions or those with atrophy, constituting a tool of great interest in monitoring and diagnosis of glaucomatous disease and other optic diseases affecting vascularisation. This method also allows taking into account the effect of loss of transparency of the lens in patients with cataracts, as this effect is compensated without affecting the final results of the determination of the amount of haemoglobin (Hb).

The present invention pertains to the sectors of Ophthalmology and Optometry, more specifically to the diagnosis of diseases of the retina and optic nerve.

The main object of the present invention is a method and device for measuring the amount of haemoglobin in the optic nerve head, of particular application to monitoring and diagnosis of glaucomatous disease.

### BACKGROUND OF THE INVENTION

It is currently known that glaucoma mainly acts through the influence of intraocular pressure on the perfusion of the optic nerve. Currently, in vivo oximetry measurements have been limited to isolated experiments using imaging spectroscopy and are based on monochromatic systems or well-known spectral composition methods in illumination or in detection. That is, they are in the field of spectrophotometry.

Spectral analysis of the image of the optic disc or optic nerve head has been mainly used to measure “oxygenation” of the haemoglobin contained. These techniques for measuring oxygen saturation in the blood vessels of the retina and optic nerve head have been widely known in the current state of the art for many years. These current techniques are based on measuring the reflected light in specific regions of the spectrum in which absorption of oxidized and reduced haemoglobin are identical (isosbestic points) and comparing the result with the absorption in other regions where there is a difference in absorption.

More specifically, Hickam et al. (1959) used 510 and 640 nm filters to calculate the ratio between the two reflected images (640/510). Later, Laing et al. (1975) used filters with wavelengths of 470-515 nm and 650-805 nm and digitised the photographic negatives with a densitometer. Subsequently, Delori (1988) used three wavelengths (558, 569 and 586 nm) and Kock et al. (1993) used 660 and 940 nm LED devices and calculated the 940/660 ratio.

The researchers Beach et al. (1999) and Crittin et al. (2002) used the 600/569 ratio to measure oxygen saturation after subtracting the background. Schweitzer et al. (1999) placed a spectrograph of images in front of the CCD sensor of a retinograph (fundus camera) to obtain spectra of arteries and veins in the retina.

Also, Bahram Khoobehi et al. (WO2005/092008) used analysis of different points of the spectrum, mainly between 545 and 570 nm (hyperspectral analysis), to measure changes in oxygen saturation in vessels of the optic nerve and tissue from animals under anaesthesia. The same method applied to human beings in vivo, by the use of monochromatic sequential illuminations obtained using a tuneable filter, was developed by Baptista et al. (2006) and Dennis et al. (2011).

All these previously cited spectral analysis methods have the drawback that they disregard the specific value of the volume of blood (amount of haemoglobin) in which oxygen saturation was evaluated and therefore the results obtained do not directly reflect the volume of blood on which the measurement is made. That is why the images obtained with these methods are not able to clearly distinguish the poorly irrigated regions (regions of atrophy or regions with little vascularised tissue) from well irrigated regions such as the neuroretinal ring.

It should be noted in relation to the previous point that tissue perfusion (oxygen supply to tissues) undoubtedly depends blood volume, to the same extent or even more than its degree of oxygenation. Therefore, these existing methods do not allow complete and adequate information of optic nerve perfusion, so it is necessary to evaluate not only the degree of oxygen saturation of haemoglobin, but also the amount of haemoglobin present in the optic nerve.

### DESCRIPTION OF THE INVENTION

The present invention resolves the previously cited drawbacks, providing a method and device for measuring the amount of haemoglobin in the optic nerve tissue and more specifically in the optic nerve head, through which it is possible to identify and clearly differentiate the irrigated regions of the optic nerve from the non-irrigated regions, this thereby constituting an extremely useful and interesting tool in monitoring and diagnosing glaucomatous disease or glaucoma. In addition, this method enables taking into account the effect of loss of lens transparency in patients with lens ageing or “cataracts”, so that the effects of spectral absorption and light diffusion are compensated without affecting the final results of the estimation and amount of haemoglobin.

The method for measuring haemoglobin (Hb) “ex vivo” in an individual, the object of the invention, is mainly based on the identification of primary colours red, green and blue (R, G, B) contained in digital images of the fundus of the eye obtained using specialised camera (fundus camera or radiograph). Through this identification of colours, it is possible to determine the amount of haemoglobin in different parts of the optic nerve such as arteries, veins, neuroretinal ring, etc., finally establishing a direct relation between the chromatic characteristics of the optic nerve tissues and the amount of haemoglobin present in the optic nerve head, thereby enabling the diagnosis of the development of glaucomatous disease.

In contrast to other regions of the fundus of the eye, the optic nerve head only contains a single pigment in significant amounts that gives it its characteristic red colour, this pigment being the haemoglobin in the vessels. Another important feature is that the tissue of the optic nerve head is a relatively thin layer on a white background constituted by “myelin”, which surrounds the axons of the nerve fibres behind the lamina cribrosa. That is why the colour of the optic nerve head essentially depends on the haemoglobin that it contains.

Moreover, the optic nerve head has two types of vessels: large vessels that are branches of the central artery and vein of the retina, which irrigate it and have no relation to nutrition of the nerve; and a network of small and capillary vessels, present in the rest of the optic nerve tissue, and which is responsible for its nutrition and oxygenation.

Therefore, the red colour of the haemoglobin depends on its preferential absorption of short wavelength light and its lower absorption of the long wavelengths of the visible spectrum. A higher amount of haemoglobin is especially manifested as increased absorption of short wavelength light. It should be clarified that although the total absorption of some wavelengths of light (red) by haemoglobin is reduced, absorption is different for oxidised and reduced haemoglobin, which enables them to be distinguished, as will be seen below.

The equipment for capturing images of the fundus of the eye (photographic or video camera) using various technologies (CCD, CMOS, etc.) measure the amount of reflected light at various wavelengths of the visible spectrum. For example, using a detector that captures three images or three chromatic components in the same image: one focussed on blue (B), another on green (G) and a third on red (R), it can be seen that in regions of high haemoglobin concentration the light reflected is mainly red (R), much less green (G) and still less blue (B). By contrast, in regions with low haemoglobin (Hb) concentration, a particularly significant increase is observed in light reflecting in the green (G) and blue (B).

More specifically, the method of measuring haemoglobin (Hb) of the present invention comprises the following stages:
identifying by colorimetry of a colour image captured of the fundus of the eye of the optic nerve head, identifying the central vessels of the retina of the optic nerve and the tissue of the eye itself that surrounds them;
establishing a first measurement, as a measurement of the amount of red light reflected (R) in the central vessels of the retina on their passage through the optic nerve head, establishing this amount as the maximum reference value of haemoglobin (Hb);
establishing a second measurement, as the measurement of the amount of red light (R) reflected in the tissue of the eye itself; and
comparison of both measurements for the determination of the amount of haemoglobin (Hb) present in the tissue of the optic nerve head, this quantity being obtained as a ratio of the first measurement compared to the second measurement.

According to a preferred embodiment, identification by colorimetry of stage a) is carried out by a software program based on MATLAB.

The first and second measurements are also preferably obtained from the frequency histograms of the captured colour image. These measurements preferably comprise the intensity of reflected light of each of the primary colours, red (R), green (G) and blue (B).

It is envisioned that the second measurement can be carried out either by frequency histogram or by spectral analysis of each point of the captured image, pixel by pixel.

According to another object of the invention, a device for measuring haemoglobin (Hb) is described below, which fundamentally comprises:
means of capturing colour images of the fundus of the eye of the optic nerve,
means of identifying the central vessels of the retina of the optic nerve and of the tissue of the eye that surrounds these vessels,
means of measuring the amount of red (R) reflected light in the central vessels of the retina and in the tissue in its passage through the optic nerve head,
means of comparing the amount of light reflected in the vessels and the amount of light reflected in the tissue, and
means of estimating the amount of haemoglobin (Hb) in the tissue of the optic nerve head as a ratio of the measurements of the amount of reflected light from the central vessels and the tissue of the retina in its passage through the optic nerve head.

Therefore, in contrast to the “spectral” analysis methods used for oximetry, the method of the present invention is based on a “colorimetric” analysis that does not require prior knowledge of the spectral composition of the incident light used in taking the images. Nor is it necessary to know the spectral curves of capture of the photographic camera used, nor to use specific wavelength of the isosbestic points, with the advantages of usability, operability and applicability that this implies.

Finally, the results of this method can be complemented by ungual oximetry in order to obtain an estimate of overall perfusion efficacy (blood volume and oxygenation).

Therefore it is noteworthy that the present invention provides a method for measuring haemoglobin in the optic nerve head that is able to compensate for different variables that influence the colour of an image of the optic nerve such as the intensity and spectral composition of the illumination system, changes in absorption at different wavelengths caused by age of the lens, its effect of diffusing the light and the spectral sensitivity properties of the detecting equipment used.

### DESCRIPTION OF THE FIGURES

To complement the description and in order to aid comprehension of the characteristics of the invention in accordance with a preferred example of a practical embodiment, this description is accompanied by a set of figures where, for the purposes of illustration and without limitation, the following are shown:

FIG. 1. Schematic view showing a section of the optic nerve head.

FIG. 2. Shows a schema of the image of the optic nerve, as observed in an image of the fundus of the eye of a patient obtained by retinography, showing the types of vessels in the optic nerve head.

FIGS. 3A, 3B, 3C, 3D. Frequency histograms showing the chromatic characteristics of the primary colours red, green, blue (R, G, B) in various optic nerve tissues.

FIGS. 4A, 4B. Frequency histogram showing the effect of diffusion and absorption by the lens on light reflected by the central vessels of the retina in is passage through the optic nerve head before operating on cataracts and the same histogram after the operation when the lens effect has been suppressed.

FIGS. 5A, 5B. Frequency histograms showing the effect of diffusion and absorption by the lens on light reflected by the tissue of the optic nerve before operating for cataracts and the same histogram after the operation when the effect of the lens with cataracts has been removed.

### PREFERRED EMBODIMENT OF THE INVENTION

An example of preferred embodiment, referring to the figures listed above, is described below, without this implying any limitation on the scope of protection of the present invention.

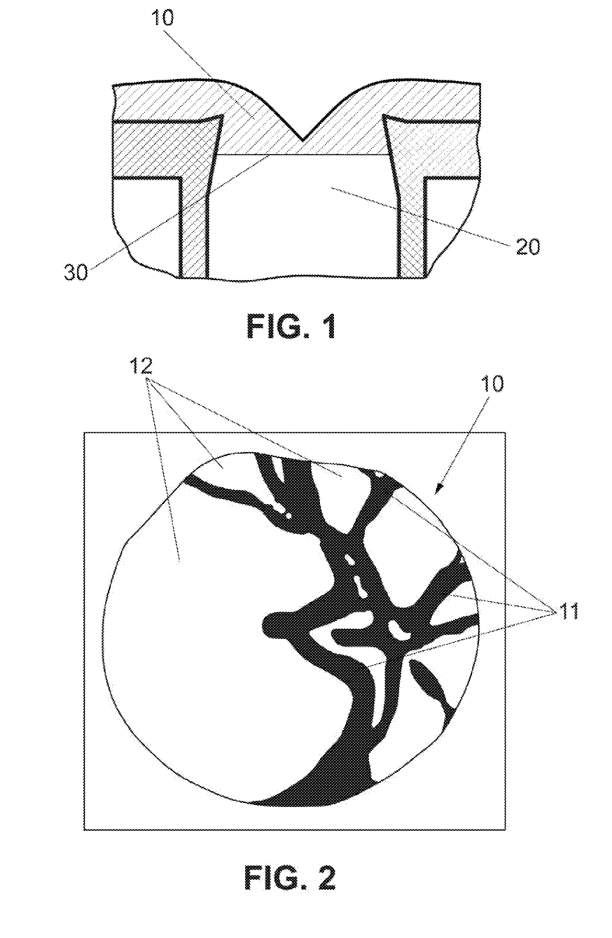

Specifically, FIG. 1 shows the tissue of the optic nerve (10) head constituted by a thin film arranged over another whitish layer that contains myelin (20) and that surrounds the axons of the optic nerve (10) fibres, when these cross the lamina cribrosa (30) in their passage towards the brain. Therefore the colour of the optic nerve head (10) fundamentally depends on the amount of haemoglobin (Hb) that it contains. In turn, the red (R) colour characteristic of haemoglobin (Hb) is due to its greater absorption of short wavelength light and its lower absorption of longer wavelengths of the visible spectrum, the colours green (G) and blue (B).

FIG. 2 schematically shows an image of the optic nerve head (10) as can be observed in the fundus of the eye of an individual, captured by specialised photographic equipment. In this image of the fundus of the eye, there are two types of vessels in the optic nerve head (10): some large vessels (11) that are branches of the central vein or artery of the retina, the function of which is mainly irrigation of the retina, and which is not related to nutrition of the optic nerve (10); and a network of fine vessels (12) and capillaries, present in the whole of the rest of the optic nerve (10) tissue and which supply nutrition and oxygenation.

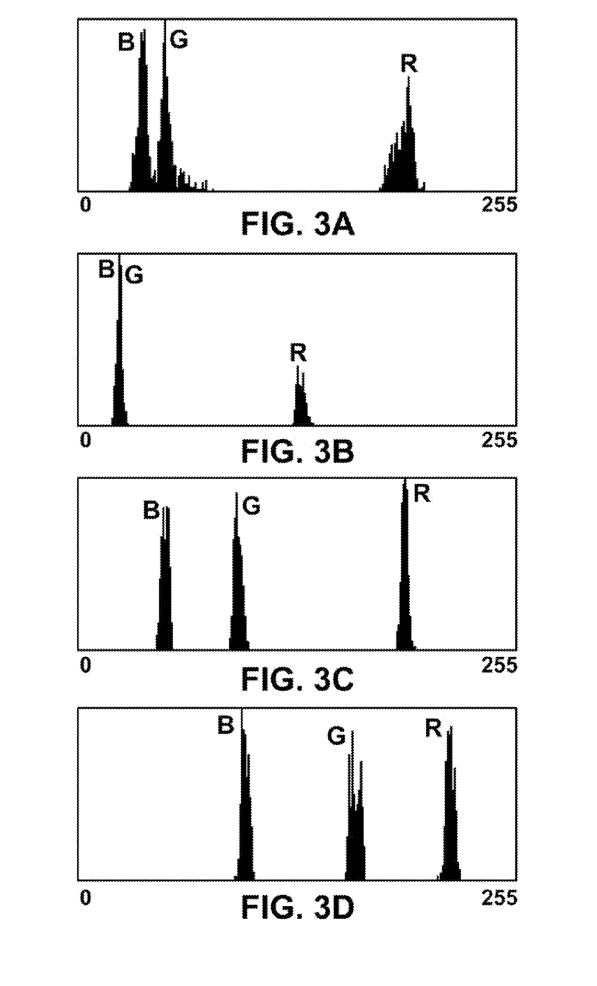

More specifically, through the frequency histogram corresponding to this image of the fundus of the eye, it is possible to determine the different intensities of reflected light for the three primary colours, red (R), green (G) and blue (B). In the present invention, the histograms obtained were made at a scale of 0 to 255, as shown in FIGS. 3A to 5B, with 0 being the value of the minimum light intensity and 255 being the maximum. These histograms show specific characteristics of each zone of the image: in the histogram of the arteries shown in FIG. 3A, there is much red (R) light reflected, much less green (G) and less blue (B). By contrast, the veins reflect less red (R) light as they contain less oxygenated haemoglobin (Hb) and very little blue (B) and green (G) in almost equal amounts, as shown in FIG. 3B.

Also, the frequency histogram in FIG. 3C shows some regions of the optic nerve (10) tissue, for example the neuroretinal ring, reflect more blue (B) and green (G) than the central vessels of the retina as they have less haemoglobin (Hb). The lower the amount of haemoglobin, the greater the amount of reflected green (G) and blue (B).

Finally, FIG. 3D shows that in regions of atrophy or lack of vascularised tissue (excavation or cavity), the proportion of blue (B) and green (G) colour increases considerably, which is perceived in the image as a whitening.

It can be experimentally verified, using dilutions of erythrocytes at different concentrations or thicknesses held in a white container, that photographic images obtained in this way enable determination of the amount of haemoglobin. Operating with amounts of primary colours red, green and blue (R, G, B) obtained using image capture systems similar to those used for the fundus of the eye, it can be seen that the result of various formulae such as R−G, R−B, R−(G+B), (R−G)/R, R+B−(2G), (R−G)/G etc., are proportional, in a practically linear way, to the amount of haemoglobin (Hb).

However, to obtain absolute and reproducible values in the optic nerve (10) tissue, it is necessary to establish a reference benchmark. This requirement originates in the fact that the result of these formulae does not only depend on the amount or volume of haemoglobin (Hb) present in the nerve but also on the intensity and spectral composition of the incident light as well as on the absorption by the lens, which fundamentally affects short wavelength light (violet-blue) and to a lesser extent green. The reference value must necessarily be within the eye, as it must be subjected to the same variables.

This reference value has been found in the large central vessels (11) of the retina in their passage through the optic nerve head (10), as these vessels (11) contain haemoglobin (Hb) in amounts that, for the effects of the haemoglobin (Hb) determination in the optic nerve (10) tissue, can be considered as maximum and constant. Therefore, this representative value of the chromatic characteristics of the vessels (11) is in the lower denominator position in the equation described below.

Therefore, the amount of haemoglobin (Hb) at each point or region of the optic nerve head (10) can be measured using an identical formula for defining the chromatic properties of the tissue (12) and of the vessels (11). Thus, the amount of haemoglobin (Hb) at each point of the tissue is expressed as a proportion of one against the other; that is:

$$Amount Hb ( \% ) = \frac{1 st measurement ( tissue )}{2 nd measurement ( vessels )} * 100$$

For the results to be correct, the image must not be over- or underexposed, given that the presence of pixels saturated in the red in the tissue, in the present invention R=255, or underexposed pixels in the vessels, in the preferred embodiment B=0, can alter the results obtained. Therefore, the specialist should analyse the image immediately after its capture, to indicate to the user if the image is correct or should be repeated using a higher or lower illumination intensity as appropriate.

In practice, to do this, mathematical algorithms for segmentation of components are applied to images of the fundus of the eye captured by photographic equipment or retinography, to identify the large central vessels (11) of the retina and the papillary edge of the optic nerve (10), enabling the specialist to verify and manually correct the results obtained. In this way, two main regions in the optic nerve head (10) are defined: the large central vessels (11) and the tissue (12) of the retina, mainly composed of axons of ganglion cells and feeder vessels.

Next, the values of the arithmetic formula selected are calculated. In the present embodiment the values of the green (G) component are subtracted from the values of the red (R) component, applying the arithmetic equation R-G to the pixels of the frequency histogram corresponding to the vessels (11), the result of which is taken as the reference value for the calculation of the amount of haemoglobin (Hb) in the tissue (12).

Finally, in the pixels of the frequency histogram of the tissue (12), the difference R−G is calculated, it is divided by the R−G value of the vessels (11) and the result multiplied by 100. In this way, the changes in intensity of spectral composition of the incident light are compensated. Finally, the concentrations of haemoglobin (Hb) are represented as pseudo-colour images, frequency histograms, average sector concentrations, etc.

In addition to all the above, an important factor to take into consideration is compensation for the change of colour caused by the lens in images of the fundus of the eye. This is because as the lens gets older it mainly absorbs radiations of short wavelength, blue (B) and to a lesser extent green (G). As this change affects both the chromatic properties of the vessels (11) and those of the tissue (12), this should not significantly alter the calculation of the amount of haemoglobin (Hb). However, the loss of transparency of the lens, as a cataract forms, also causes an increase of the diffusion of light in the images of the fundus of the eye, which increases the green (G) component of the vessels (11) with light coming from the tissue (12).

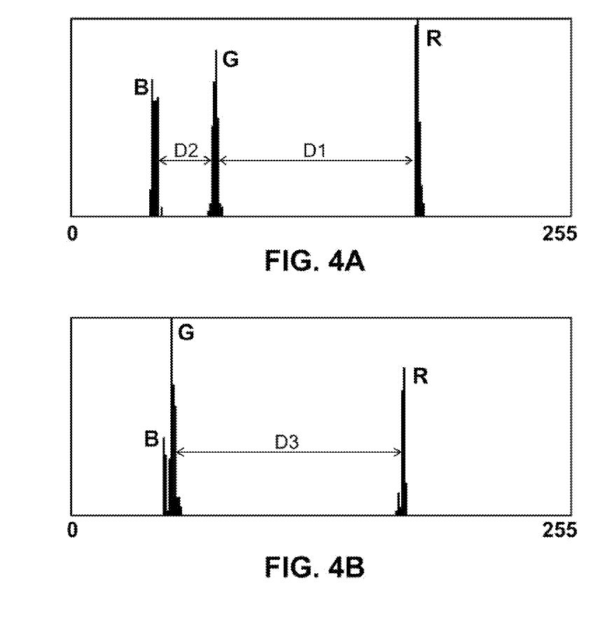

More specifically, FIG. 4A shows the frequency histogram of the vessels (11) of a patient with cataracts. In this FIG. 4A, the distance (D1) between the red (R) and green (G) components is shortened, along with the distance (D2) between the blue (B) and green (G) components being lengthened. FIG. 4B shows the frequency histogram after operating for the patients cataracts. This FIG. 4B shows an increase in the value of the blue (B) component when removing the absorption of the lens, reducing the green (G) component as a consequence of less diffusion, so that the distance (D3) between the red (R) and green (G) components increases. It should be remembered that when speaking here of an increase or reduction of the components of the primary colours (R, G, B), this is relative to the horizontal axis of the frequency histograms.

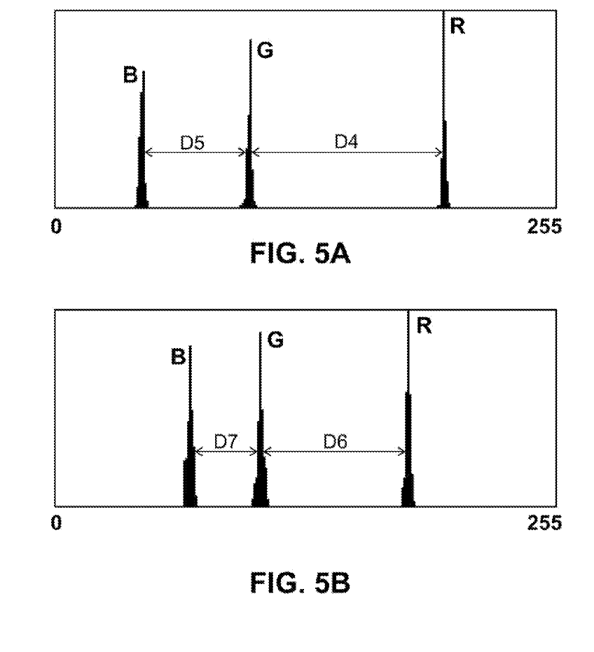

FIGS. 5A and 5B show that in frequency histograms of the tissue (12), an equivalent phenomenon occurs. In the frequency histogram of the patient with cataracts, see FIG. 5A, the diffusion and absorption of the green (G) and blue (B) components increases the distances (D4, D5) between the red (R) and green (G) components and between the green (G) and blue (B) components respectively, also contributing to an increase in the increment distance (D4) of the red (R) component caused by the diffusion of light from the vessels. If not corrected, the relative reddening of the image causing this problem can lead to an over-estimation of the amount of haemoglobin (Hb) in the tissue.

The frequency histogram of FIG. 5B shows that when operating on the patient with cataracts, the distances (D6, D7) between the red (R) and green (G) components and between the green (G) and blue (B) components decrease compared to the equivalent distances (D4, D5), so that the region appears whiter.

As both effects of the lens, on the vessels (11) and on the tissue (12), are proportional, measuring the distance (D2) between the green (G) and blue (B) in the frequency histogram of the vessels (11) can lead to an estimation of the degree of this absorption-diffusion effect of the lens on the tissue (12), which enables compensating for its effect on the estimation of the amount of haemoglobin (Hb).

1. Method of measuring haemoglobin (Hb) “ex vivo” of an individual characterised in that it comprises the following stages:
a) identification of the optic nerve head (10) using colorimetry of a colour image captured of the fundus of the eye, identifying the central vessels (11) of the retina of the optic nerve (10) and the tissue (12) of the eye that surrounds them;
b) establishing of a first measurement, as a measurement of the amount of red light reflected (R) in the central vessels (11) of the retina on their passage through the optic nerve head (10), establishing this amount as the maximum reference value of haemoglobin (Hb);
c) establishing a second measurement, as the measurement of the amount of red (R) reflected light in the tissue (12) of the eye itself; and
d) comparing both measurements for the determination of the amount of haemoglobin (Hb) in the tissue (12) of the optic nerve head (10), this amount being obtained as a ratio of the second measurement compared to the first measurement.

2. Method of measuring haemoglobin (Hb), according to claim 1, characterised in that the colour image of the fundus of the eye of the optic nerve (10) is captured by specialised photographic equipment.

3. Method of measuring haemoglobin (Hb), according to claim 1, characterised in that the identification by colorimetry of stage a) is carried out by a software program based on MATLAB.

4. Method of measuring haemoglobin (Hb), according to claim 1, characterised in that the first measurement and the second measurement are obtained from frequency histograms of the captured colour image.

5. Method of measuring haemoglobin (Hb), according to claim 1, characterised in that the intensities of reflected light for each of the primary colours red (R), green (G) and blue (B) are obtained when carrying out the first and the second measurements.

6. Method of measuring haemoglobin (Hb), according to claim 1, characterised in that the second measurement is carried out through a frequency histogram.

7. Method of measuring haemoglobin (Hb), according to claim 1, characterised in that the second measurement is carried out through a specific analysis of each point of the captured image, pixel by pixel.

8. Method of measuring haemoglobin (Hb), according to claim 5, characterised in that the determination of the amount of haemoglobin (Hb) in the tissue (12) of the optic nerve head (10) is obtained using the formula:
$$Amount Hb ( \% ) = \frac{1 st measurement ( tissue )}{2 nd measurement ( vessels )} * 100$$

9. Device for measuring haemoglobin (Hb) characterised in that it comprises:
means of capturing colour images of the fundus of the eye,
means of identifying, in said captured images, the central vessels (11) of the retina of the optic nerve (10) and the tissue (12) of the eye that surrounds these vessels (11),
means of measuring the amount of red (R) reflected light in the central vessels (11) of the retina and in the tissue (12) in its passage through the optic nerve head (10),
means of comparing the amount of light reflected in the vessels (11) and the amount of light reflected in the tissue (12), and
means of estimating the amount of haemoglobin (Hb) in the tissue (12) of the optic nerve head (10) as a ratio obtained dividing the measurement of reflected light from the tissue by the measurements of reflected light from the central vessels (11), in their passage through the optic nerve head (10).
